(19) United States
(12) Patent Application Publication
(10) Pub. No.: US 2023/0128500 A1
(43) Pub. Date: Apr. 27, 2023

(54) TARGET APERTURED TOPSHEET

(71) Applicant: ASSOCIATED HYGIENIC PRODUCTS LLC, Raleigh, NC (US)

(72) Inventor: Trenton OTTERY, Delaware, OH (US)

(73) Assignee: ASSOCIATED HYGIENIC PRODUCTS LLC, Raleigh, NC (US)

(21) Appl. No.: 18/045,587

(22) Filed: Oct. 11, 2022

Related U.S. Application Data
(60) Provisional application No. 63/270,441, filed on Oct. 21, 2021.

(51) Int. Cl. A61F 13/537; A61F 13/512; A61F 13/513

(52) U.S. Cl. CPC A61F 13/53747; A61F 13/5123; A61F 13/51305

(57) ABSTRACT

The present disclosure includes absorbent products having an apertured zone in the surface facing the wearer for enhanced absorbency and BM containment. Some aspects of the products include a chassis including a topsheet defining a plurality of apertures, and a backsheet, an absorbent core disposed between the topsheet and the backsheet, and an acquisition-distribution layer (ADL) disposed between the topsheet and the absorbent core, the ADL including a plurality of external edges that cooperate to define an ADL boundary. In some aspects at least 75% of the plurality of apertures are defined within an apertured region that is disposed vertically above the ADL and is enclosed within the ADL boundary when the chassis is in a flat configuration.

### CROSS-REFERENCE TO RELATED APPLICATIONS

This application claims priority to and the benefit of U.S. Provisional Application No. 63/270,441, filed Oct. 21, 2021, the contents of which are incorporated into the present application by reference in its entirety.

### FIELD OF INVENTION

The present invention relates generally to absorbent garments and, more particularly, to diapers having an apertured zone in the surface facing the wearer for enhanced absorbency and bowel movement (BM) containment.

### BACKGROUND

Absorbent products, such as baby diapers, training pants, adult incontinence briefs and underwear, all of which may be made in disposable forms. “Disposable” refers to articles that are designed to be discarded after a limited use rather than being laundered or otherwise restored for reuse. Disposable absorbent products have met with widespread acceptance in the marketplace for a variety of applications, including infant and adult incontinence care, in view of the manner in which such products can provide effective and convenient liquid absorption and retention while maintaining the comfort of the wearer. Such disposable absorbent articles often include a topsheet that is configured to be closest to the wearer during use, a liquid-impermeable backsheet or outer cover, and an absorbent core between the topsheet and the backsheet. Conventional absorbent cores often include superabsorbent polymer or “SAP,” such as hydrogel-forming polymer material. The inclusion of SAP in an absorbent core typically also increases the ability of the absorbent core, relative to a similarly sized absorbent core of fluff pulp alone, to retain absorbed liquid against pressure, thus providing lower rewet and better skin dryness. In some instances, such disposable absorbent articles also include an acquisition-distribution layer (ADL) disposed between the topsheet and the absorbent core.

Typical topsheet materials are coated with a surfactant to make them hydrophilic. Such materials are largely adequate in allowing urine to pass through to the absorbent core. However, these material do little to help bowel movement (BM) containment. Particularly, a runny BM, which includes a fluid-solid mixture that does not easily pass through the topsheet and, thus, will have the tendency to run across the surface of the topsheet. Further, the fluid-solid mixture may be unable to be absorbed by the article and may find its way outside of the product, referred to as a leak or “blowout.” There are two approaches commonly used by absorbent products to increase BM containment and decrease chances of a blowout.

The first approach involves providing an apertured topsheet to increase the ability of the fluid-solid mixture associated with a runny BM to pass through the topsheet and be absorbed by the core, or simply creating a 3-dimensional surface that makes it more difficult for fluid to travel across the surface. Current articles have topsheets that are fully-apertured such that the apertures cover an entire surface of the topsheet. While this is helpful in BM containment, there are also disadvantages of having an apertured topsheet. Namely, the topsheet also functions to keep the SAP in the core from escaping up through the topsheet and getting on the wearer. Thus, while apertured topsheets enable passage of fluid-solid mixture to the core, these topsheets also enable passage of SAP from the core through the topsheet. Accordingly, if the topsheet is apertured then the design must account for another way to contain the SAP in the core.

The second approach is to include a BM containment feature such as a pocket, pouch, voids, or other feces containment portion in the back of the article to contain the BM and stop fluid-solid mixture flow. While such features increase BM containment, they also increase design complexity which results in increased manufacturing time and price. Additionally, these features are inadequate at containing excess fluid associated with runny BMs from leaking across the topsheet into a front portion of the absorbent product.

### SUMMARY

The present absorbent garments provide improved BM containment with a target apertured topsheet. Some of the present absorbent garments include a chassis having opposing front and rear waist portions, a crotch portion extending longitudinally between the front and rear waist portions, a topsheet defining a plurality of apertures, and a backsheet. Such garments may include an absorbent core disposed between the topsheet and the backsheet, at least a portion of the absorbent core positioned in the crotch portion and an acquisition-distribution layer (ADL) disposed between the topsheet and the absorbent core, the ADL including a plurality of external edges that cooperate to define an ADL boundary. In some aspects, substantially all of the plurality of apertures are defined within an apertured region that is disposed vertically above the ADL and is enclosed within the ADL boundary when the chassis is in a flat configuration. For example, at least 75%, such as 80, 85, 90, 95, 98, or 100%, of the plurality of apertures defined by the topsheet are positioned within the apertured region. The present topsheet may include a topsheet that is only apertured within a targeted region to increase fluid absorption for BM containment as well as prevent SAP leakage. Accordingly, the absorbent garments described herein can help with BM containment in an area that it is most needed and there is no longer a need for an alternative methods of SAP containment.

In some garments, the apertured region overlies at least a majority of the ADL. Alternatively, in other garments, the apertured region overlies an entirety of the ADL. In some of the forgoing garments, a width and a length of the apertured region are substantially equal to a width and a length, respectively, of the ADL. In some configurations, the external edges of the ADL includes two widthwise edges extending in a first direction; and two lengthwise edges extending in a second direction perpendicular to the first direction

In some garments, the chassis includes a first end and a second end opposite the first end. The front waist portion extending from the first end and the rear waist portion extending from the second end. In some such configurations, the apertured region is positioned closer to the first end than the second end. In some configurations, at least a majority of the apertured region is disposed in a front half of the chassis.

In some of the foregoing configurations, the topsheet includes the apertured region and a non-apertured region. In some configurations, the apertured region includes a percentage of open area greater than or equal to 5% (e.g., between 5 and 40%) and the non-apertured region includes a percentage of open area less than 2% (e.g., less than 1%). Each aperture in the apertured region may include a maximum transverse dimension that is between 0.25 and 5.0 millimeters (mm). In some configurations, the non-apertured region spans at least a majority of the topsheet. The topsheet may include only a single apertured region. In some aspects, the topsheet includes a liquid-permeable nonwoven and the backsheet includes a liquid impermeable material.

Some of the present absorbent garments include a topsheet and a backsheet, the topsheet defining a plurality of apertures and having an apertured region and a non-apertured region. Some such garments include an absorbent core disposed between the topsheet and the backsheet and an acquisition-distribution layer (ADL) disposed between the topsheet and the absorbent core, the ADL including a plurality of external edges that cooperate to define an ADL boundary. The apertured region may overlie the ADL and be enclosed within the ADL boundary. In some configurations, an area of the ADL is less than or equal to an area of the absorbent core. Additionally, or alternatively, an area of the apertured region may be between 25% and 95% of the area of the ADL. In some configurations, an area of the non-apertured region is greater than the area of the ADL. In some garments, the chassis includes a first end and an opposing second end, a front portion extending from the first end, and a rear portion extending from the second end. In some such configurations, at least a majority of the apertured region is disposed within the front portion of the chassis. In some configurations, an entirety of the apertured region is disposed within the front portion of the chassis.

As used herein, various terminology is for the purpose of describing particular implementations only and is not intended to be limiting of implementations. For example, as used herein, an ordinal term (e.g., “first,” “second,” “third,” etc.) used to modify an element, such as a structure, a component, an operation, etc., does not by itself indicate any priority or order of the element with respect to another element, but rather merely distinguishes the element from another element having a same name (but for use of the ordinal term). The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified — and includes what is specified; e.g., substantially 90 degrees includes 90 degrees and substantially parallel includes parallel—as understood by a person of ordinary skill in the art. In any disclosed embodiment, the term “substantially” may be substituted with “within [a percentage] of” what is specified, where the percentage includes 0.1, 1, 5, and 10 percent. The phrase “and/or” means and or or. To illustrate, A, B, and/or C includes: A alone, B alone, C alone, a combination of A and B, a combination of A and C, a combination of B and C, or a combination of A, B, and C. In other words, “and/or” operates as an inclusive or. Unless stated otherwise, the term “or,” as used herein, refers to an inclusive or and is interchangeable with the term “and/or.”

The terms “comprise” and any form thereof such as “comprises” and “comprising,” “have” and any form thereof such as “has” and “having,” and “include” and any form thereof such as “includes” and “including” are open-ended linking verbs. As a result, an apparatus that “comprises,” “has,” or “includes” one or more elements possesses those one or more elements, but is not limited to possessing only those elements. Likewise, a method that “comprises,” “has,” or “includes” one or more steps possesses those one or more steps, but is not limited to possessing only those one or more steps.

Any embodiment of any of the apparatuses, systems, and methods can consist of or consist essentially of—rather than comprise/include/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

Further, a device or system that is configured in a certain way is configured in at least that way, but it can also be configured in other ways than those specifically described. The feature or features of one embodiment may be applied to other embodiments, even though not described or illustrated, unless expressly prohibited by this disclosure or the nature of the embodiments. Some details associated with the embodiments described above and others are described below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. Views in the figures are drawn to scale, unless otherwise noted, meaning the sizes of the depicted elements are accurate relative to each other for at least the embodiment in the view.

FIGS. 1A and 1B are plan views of a disposable absorbent article in an open configuration.

FIG. 1C is an enlarged plan view of portion 1C of the disposable absorbent article of FIG. 1B.

FIG. 1D is an enlarged plan view of a topsheet of FIG. 1B and illustrates the dimensions and spacing of the topsheet's apertures.

FIG. 1E is an exaggerated partial sectional view of the garment of FIG. 1B taken along line 1E-1E.

FIG. 2 is a top plan view of a second configurations of the present disposable absorbent article.

FIG. 3 is a top plan view of a third configurations of the present disposable absorbent article.

FIG. 4 is a top plan view of a fourth configurations of the present disposable absorbent article.

FIG. 5 is a top plan view of a fifth configurations of the present disposable absorbent article.

### DETAILED DESCRIPTION OF ILLUSTRATIVE EMBODIMENTS

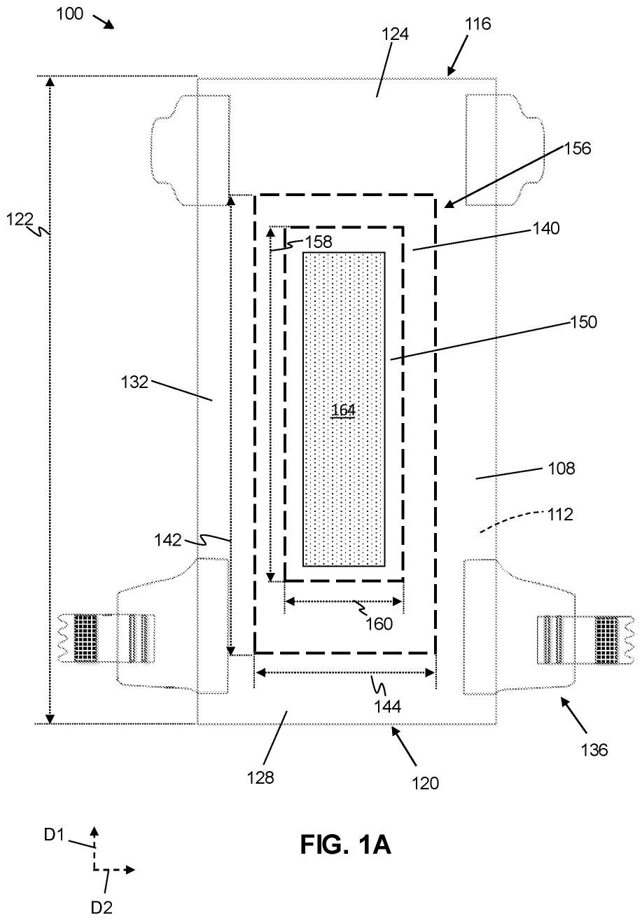

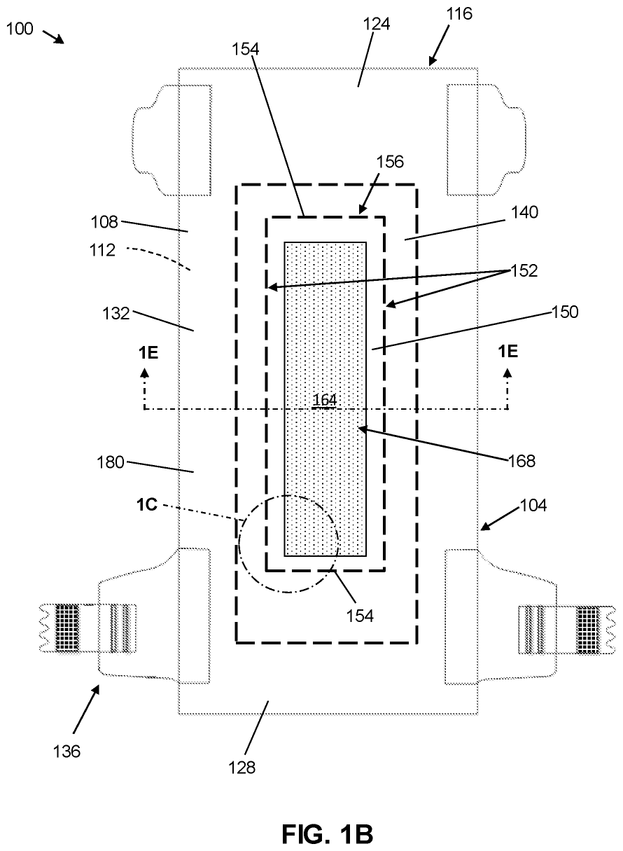

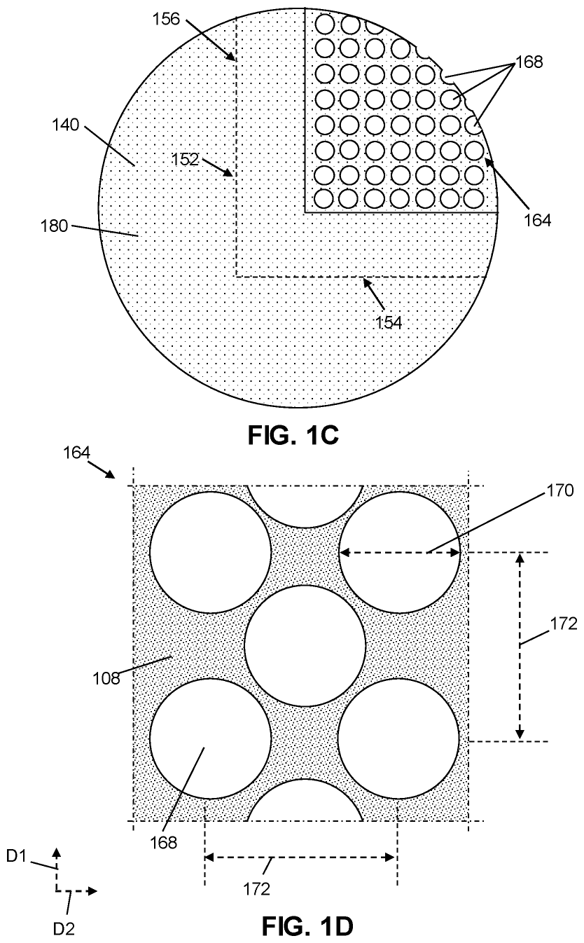

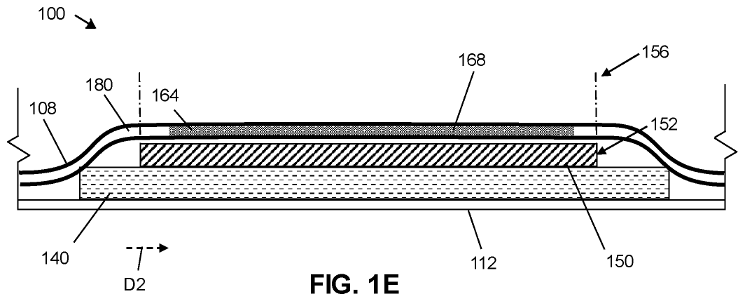

Referring to FIGS. 1A-1E, shown is an example of a configuration 100 of the present absorbent articles. Article 100 may be an underpad, a bed pad, seat cushion, patient support pad, breast pad, bandage, baby diaper, training pant, adult incontinence brief or underwear, bladder control pad, feminine hygiene pad, or the like. Article 100 includes a chassis 104 having a liquid-permeable topsheet 108 configured to face a user and a liquid-impermeable backsheet 112 configured to face away from the user during use of the article 100. For example, backsheet 112 can include an inner liquid-impermeable film and an outer nonwoven cover, and can be breathable, e.g., the liquid-impermeable film can comprise a breathable film.

As shown in FIG. 1A, chassis 104 has a first end 116 and an opposing second end 120 and a length 122 extending between the first end and the second end. Length 122 is measured in a machine or longitudinal direction D1 and may extend substantially perpendicular to first end 116, and second end 120 when chassis 104 is in a relaxed state (e.g., laid out flat without any external forces acting on the chassis). In some configurations, length 122 can be can be greater than or substantially equal to any one of, or between any two of: 200, 250, 300, 350, 400, 450, 500, or 550 millimeters (mm) (e.g., between 200 and 330 mm). In some configurations, such as articles for adults, length 122 may be greater than 550 mm.

Chassis 104 includes a front waist portion 124, an opposing rear waist portion 128, and a crotch portion 132 extending longitudinally between the front and rear waist portions. Front waist portion 124 includes a portion of chassis 104 extending from a first end 116 that is associated with a front end (e.g., edge) of article 100 when worn by a user. In some configurations, front waist portion 124 includes only first end 116, while, in other configurations, the front waist portion includes the first end and a portion of the chassis extending from the first end. In some configurations, front waist portion 124 may have a length that is less than, equal to, or between any two of: 25, 20, 15, 12, 10, 8, 6, 5, 4, 3, 2, or 1 percent of length 122 (e.g., the overall length) of chassis 104. Rear waist portion 128 includes a portion of chassis 104 extending from a second end 120 that is associated with a rear end (e.g., edge) of article 100 when worn by a user. Rear waist portion 128 includes second end 120 and/or a portion of the chassis extending from the second end. In some configurations, rear waist portion 128 may have a length that is less than, equal to, or between any two of: 25, 20, 15, 12, 10, 8, 6, 5, 4, 3, 2, or 1 percent of length 122 of chassis 104. In some configurations, crotch portion 132 includes a portion of chassis 104 extending directly between front waist portion 124 and rear waist portion 128.

As shown in FIGS. 1A and 1B, article 100 may, but need not, include a pair of closure members 136 configured to couple front waist portion 124 or to rear waist portion 128 in a well-known configuration in which a left side of chassis 104 defines a first leg opening for a wearer's left leg, and in which a right side of the chassis defines a second leg opening for the wearer's right leg. Closure members 136 (e.g., ears) may have a first end bonded to a portion of chassis 104 and a second end that extends away from the chassis. Closure members 136 can have fastener tabs to enable the closure members to latch onto another portion of article 100 (e.g., chassis 104, landing pad, another closure member, or the like) so that article 100 can define a closed configuration in which the garment is wearable. For example, in the closed configuration, front and rear waist portions 124 and 128 can cooperate to encircle and define a waist opening, a left side of chassis 104 can define a first leg opening, and a right side of the chassis can define a second leg opening. In this way, article 100 is movable from a flat configuration (shown in FIGS. 1A and 1B) in which chassis 104 is substantially disposed within a single plane (e.g., laid flat) and the closed configuration in which the article is configured to be worn by a wearer.

In some configurations, article 100 includes an absorbent core 140 disposed between topsheet 108 and backsheet 112. Core 140 is configured to absorb liquid in the absorbent article and may include materials suitable for absorbing liquids, such as, for example, a mixture of conventional fluff (e.g., cellulosic fibers) and superabsorbent polymer (SAP) particles. “Superabsorbent” or “superabsorbent material” or “SAP” refers to a water-swellable, water-insoluble organic or inorganic material capable, under the most favorable conditions, of absorbing at least about 15 times its weight in an aqueous solution containing 0.9 weight percent sodium chloride and, more desirably, at least about 30 times its weight in an aqueous solution containing 0.9 weight percent sodium chloride and, even more desirably, at least about 50 times its weight in an aqueous solution containing 0.9 weight percent sodium chloride.

Article 100 also includes an acquisition-distribution layer (ADL) 150 disposed between the topsheet and the absorbent core. “Layer” when used in the singular can be a single element or a plurality of elements. For example, a plurality of sheets may together define a single layer, such as, for example, a layer with a particular function to which the sheets of the layer contribute. ADL 150 functions to acquire the liquid as rapidly as it is added to the product (to prevent uncontrolled surface runoff) and then spreads or distributes the acquired liquid over a larger surface area of absorbent core 140 at a rate that the core can absorb the liquid without undesired internal runoff and leakage. The core 140, in turn, wicks the liquid into the core to reduce wetness in ADL 150, to present a relatively dry ADL surface to the wearer and partially restoring the ADL to its initial state to acquire subsequent insults of liquid.

ADL 150 can be sized and have a construction to help mitigate liquid trapping in topsheet 108 and thereby promote dryness at the topsheet. For example, ADL 150 can include one or more longitudinal edges 152 and one or more lateral edges 154 that cooperate to define an ADL boundary 156. Longitudinal edges 152 include edges of ADL 150 which extend further in longitudinal direction D1 than lateral direction D2 and lateral edges 154 include edges of the ADL which extend further in the lateral direction than the longitudinal direction D1. As shown in FIG. 1, ADL 150 includes two longitudinal edges 152 and two lateral edges 154, yet, in other configurations, the ADL may include more than two longitudinal edges or more than two longitudinal edges. In some configurations, longitudinal edges 152 extend in a direction substantially parallel to length 122 (e.g., parallel to longitudinal direction D1). Additionally, or alternatively, lateral edges 154 may extend in a direction substantially perpendicular to length 122 (e.g., parallel to lateral direction D2).

In some configurations, an area of ADL 150 (e.g., surface of ADL enclosed by ADL boundary 156) might be smaller than absorbent core 140; in this manner, the larger absorbent core can have a relatively large absorption capacity and the smaller ADL can require fewer materials for its construction (e.g., to reduce costs) while being positioned at or near the location on chassis 104 likely to received liquid insults (e.g., closer to front waist portion 124 than to rear waist portion 128) such that it adequately promotes topsheet dryness. In the illustrative example shown in FIG. 1A, a length 158, as measured in a machine or longitudinal direction D1, of ADL 150 can be less than or equal to any one of, or between any two of, 90%, 80%, 70%, 60%, 50%, or 40%, 30% of length 142 of absorbent core 140 and a width 160, as measured in a cross- or transverse dimension D2 that is perpendicular to the machine/longitudinal direction, of the ADL can be less than or equal to any one of, or between any two of, 90%, 80%, 70%, 60%, 50%, or 40% of the width 144 of the absorbent core. For some articles, length 158 can be greater than or equal to any one of or between any two of 140, 150, 160, 170, 180, 190, 200, 210, 220, 230, 240, 250, 260, 270, or 280 millimeters (mm) (e.g., between 185 and 270 mm) and width 160 can be greater than or equal to any one of, or between any two of, 50, 55, 60, 65, 70, 75, 80, 85, 90, 95, 100, or more millimeters (e.g., between 65 and 85 mm, or 75 mm). In other embodiments, ADL 150 can span substantially the same area as absorbent core 140 such that length 142 of the absorbent core is within 10% of length 158 of the ADL and/or width 144 of the absorbent core is within 10% of width 160 of the ADL.

Article 100 further includes one or more apertured regions 164 in which topsheet 108 defines a plurality of apertures 168 and one or more non-apertured regions 180. In some configurations, apertured region 164 includes a portion of topsheet 108 having an aperture density (e.g., as determined by dividing the total number of apertures 168 by an area of the topsheet) is greater than or equal to any one of, or between any two of: 1.0, 1.5, 2.0, 2.5, 3.0, 3.5 4.0, 4.5, 5.0, 6.5, 7.0, 7.5, 8.0, 8.5, 9.0, 9.5, 10.0, 10.5, 11.0, 11.5, 12.0, or 15.0 (e.g., between 5 and 12) apertures 168 per square centimeter of topsheet 108. Additionally, or alternatively, apertured region 164 includes a portion of topsheet 108 having a percentage of open area (e.g., as determined by dividing a total area of apertures 168 by an area of the topsheet) that is greater than or equal to any one of, or between any two of: 5, 10, 15, 20, 25, 30, 35, 40, 45 or 50 percent (%) (e.g., between 10 and 30% or between 15 and 25%. As depicted, substantially all of (e.g., at least 75% of, and preferably at least 80, 90, 95, 98, or 100% of) the plurality of apertures 168 defined by topsheet 108 are disposed within apertured region 164 Apertures 168 can be arranged in any suitable manner, such as, for example, in a plurality of rows and columns that may be aligned (e.g., a grid as shown in FIG. 1C) or offset from one another (e.g., as shown in FIG. 1D). In yet other configurations, may be arranged in another pattern or randomly disposed throughout apertured region 164. In other configurations, apertures 168 may be arranged to define a design or graphic.

Each of apertures 168 can have any suitable shape and size; as shown in FIG. 1D, each of the apertures has a convex surface and can have a circular cross-section. In other configurations, apertures 168 may have any other suitable geometry, such as circular, elliptical, otherwise rounded, or triangular, rectangular, hexagonal, otherwise polygonal, or the like. In some configurations, apertures 168 can be relative large such that apertured region 164 has a small aperture density (e.g., 5.0 or less) and a large percentage of open area. In other configurations, apertures 168 can be relative small such that apertured region 164 has a large aperture density (e.g., 10.0 or more) of apertured region and a large percentage of open area. In an illustrative example, each aperture 168 can have a maximum transverse dimension 170 (e.g., diameter) that is greater than or equal to any one of, or between any two of, 0.25, 0.5, 1.0 1.5, 2.0, 2.5, 3.0, 3.5, 4.0, 4.5, 5.0, 5.5, 6.0, 6.5, 7.0, 7.5, 8.0, 8.5, or 9.0 mm (e.g., between 0.5 and 5 mm). The dimensions of apertures 168 can be the same or can be varied. To illustrate, the maximum transverse dimension 170 of each aperture may be substantially the same (e.g., within 5% of each other) while in other configurations, the maximum transverse dimension of at least one aperture may be greater than (e.g., at least 5% greater than) the maximum transverse dimension of one other aperture.

Apertures 168 can be arranged in two or more rows, each of the rows including two or more of the apertures aligned in lateral direction D2. Additionally, or alternatively, Apertures 168 can be arranged in two or more columns, each of the columns including two or more of the apertures aligned in longitudinal direction D1. In some configurations, apertures 168 may, but need not, be equally spaced. In some such configurations, each aperture 168 can be spaced apart from at least one other of the protrusions by a distance 172, measured between the centers of the apertures, that is greater than or equal to any one of, or between any two of, 1.0, 1.5, 2.0, 3.0, 3.5, 4.0, 4.5, 5.0, 5.5, 6.0, 6.5, 7.0, 7.5, 8.0, 8.5, 9.0 or 10.0 mm (e.g., between 1.0-6.0 mm).

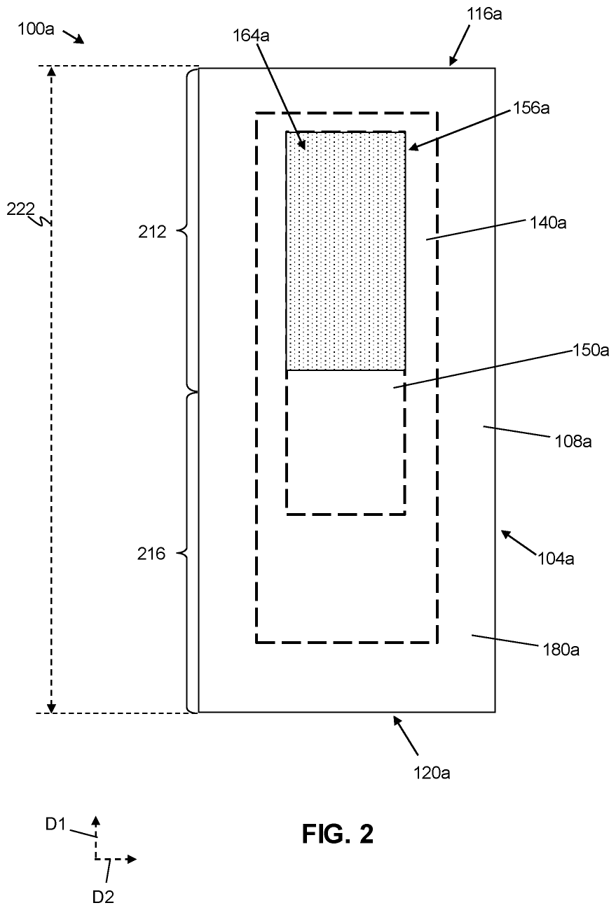

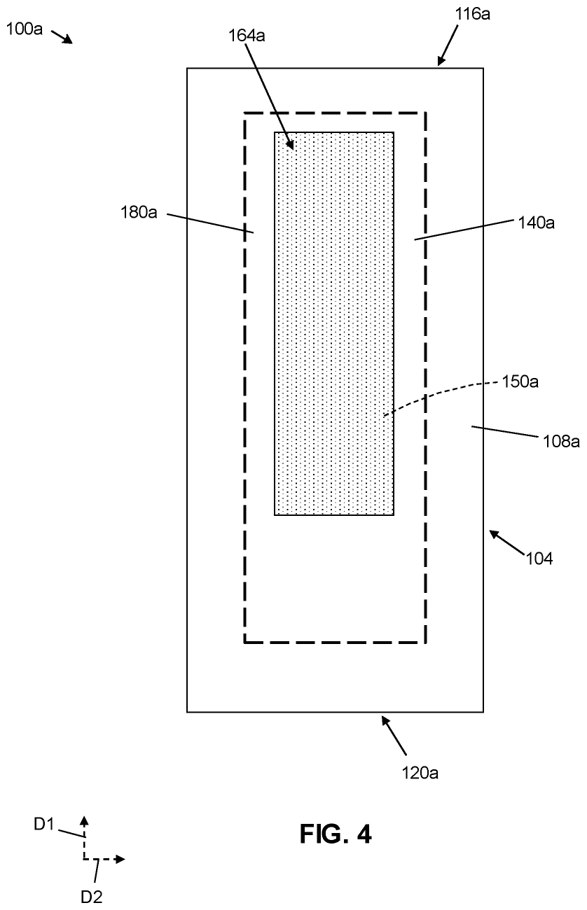

Apertured region 164 overlies at least a portion of ADL 150. In some configurations, apertured region 164 is contained to portions of topsheet 108 that overlie ADL 150. For example, as shown in FIG. 1E, apertured region 164 is vertically disposed above ADL 150, when chassis 104 is in the flat configuration. In some configurations, apertured region 164 is enclosed within the ADL boundary, when chassis 104 is in a flat configuration. To illustrate, apertured region 164 is disposed between longitudinal edges 152 when viewed along a plane parallel to lateral direction D2 (as shown in FIG. 1E) and disposed between lateral edges 154 when viewed along a plane parallel to longitudinal direction D1. Accordingly, topsheet 108 may only be apertured inside ADL 150 (e.g., apertured region 164 is only disposed within ADL boundary 156). Apertured region 164 may overlie only a portion of ADL 150 or may overlie substantially an entirety of the ADL, as described further with reference to FIGS. 2-5. In this way and others, liquid passed through apertures 168 of topsheet 108 is delivered to ADL 150 where the liquid may be spread throughout the ADL for steady absorption to core 140.

In some configurations, apertured region 164 may be at least partially disposed within front waist portion 124 or crotch portion 132 to limit fluid (e.g., from a runny BM) flow across topsheet 108 toward the front waist portion. In some such configurations, apertured region 164 may be disposed nearer to front waist portion 124 (e.g., first end 116) than to rear waist portion 128 (e.g., second end 120). In some configurations, apertured region 164 is not positioned within rear waist portion 128. In other configurations, an area of apertured region 164 positioned within front waist portion 124 is greater than an area of the apertured region positioned within rear waist portion 128.

Non-apertured region 180 includes a portion of topsheet 108 having an aperture density less than 2.0 apertures 168 (e.g., less than 1.5, 1.0, 0.5, or 0.25 apertures) per square centimeter of topsheet 108, a percentage of open area that is less than 30% (e.g., less than 25, 20, 15, 10, 5, 3 or 1%), or both. Accordingly, non-apertured region 180 may not enable a fluid (or a fluid-solid mixture) to easily pass through topsheet 108. In some configurations, as shown in FIG. 1D, at least a portion of non-apertured region 180 is vertically disposed above core 140. Additionally, or alternatively, non-apertured region 180 may, but need not, be vertically disposed above a portion of ADL 150. In some configurations, non-apertured region 180 may surround apertured region 164.

Non-apertured region 180 may make up at least a majority of topsheet 108. For example, non-apertured region 180 may have an area that is between 50% and 90% (e.g., between any two of 50, 55, 60, 65, 70, 75, 80, 85, or 90%) of an area of topsheet 108. Non-apertured region 180 may abut a portion of apertured region 164 and, in some configurations, the non-apertured region and the apertured region cooperate to form an entirety of topsheet 108. In some such configurations, non-apertured region 180 may surround apertured region 164. As described herein, non-apertured region 180 may limit the amount to fluid that passes through topsheet 108. This may allow chassis 104 to (1) direct fluid flowing on topsheet to certain portions of the topsheet (e.g., apertured region 164) where the fluid may be better absorbed into ADL 150 and/or core 140, (2) limit fluid from escaping from the core up through the topsheet, or both.

Referring additionally to FIGS. 2-5, shown are additional configurations 100a of the present articles that is substantially similar to article 100, depicting alternate positioning of apertured region 164a on topsheet 108a. Article 100a includes a chassis 104a having a topsheet 108a defining a plurality of apertures (e.g., 168), a backsheet 112a, a core 140a disposed between the topsheet and the backsheet, and an ADL 150a disposed between the core and the topsheet. Substantially all of (e.g., at least 90% of) the apertures are defined in an apertured region 164a of topsheet that overlies ADL 150a. Apertured region 164a may include or correspond to apertured region 164. Although not shown for convenience, article 100a may include one or more other components (e.g., such as the components of article 100, described above).

As shown in FIG. 2, chassis 104a may include a front portion 212 and a rear portion 216. Front portion 212 includes a portion of chassis 104a extending from a first end 116a toward a second end 120a and having a length that is 50% of a length 222 of the chassis and rear portion 216 includes a portion of the chassis extending from the second end 120a toward the first end and having a length that is 50% of the length 222 of the chassis. In some configurations, such as the configuration shown in FIG. 2, apertured region 164a of article 100a is disposed entirely within front portion 212. Although apertured region 164a is shown as overlying only a portion of ADL 150a, in other configurations of article 100a, the apertured region 164a overlies substantially all of the ADL 150a (e.g., the apertured region is disposed vertically above the ADL has an area that is at least 90% of the area of ADL).

Unexpectedly, apertured region 164a may promote sufficient BM containment despite being positioned further away from rear portion 216, which is nearest the anus of a wearer when article 100a is worn. For runny BMs, the fluid-solid mixture will travel along topsheet 108a and be readily absorbed in ADL 150a via apertures (e.g., 168) and mitigate the risk of the fluid-solid mixture from leaking outside of article 100a. In some configurations, apertured region 164a is positioned near rear portion 216 (e.g., portion of front portion 212 nearest the rear portion) to absorb the fluid-solid mixture before the mixture travels further into the front portion. In this way, apertured region 164a may limit contamination of the mixture to the wearer's anus and avoid contamination of skin nearer the wearer's genitals. Additionally, as apertured region 164a does not overlie an entirety of core 140, topsheet 108a (via non-apertured region 180a) also functions to keep material (e.g., SAP) in the core from escaping up through the topsheet and getting on the wearer. Accordingly, apertured region 164a provides adequate BM containment without the additional need for an alternative SAP containment.

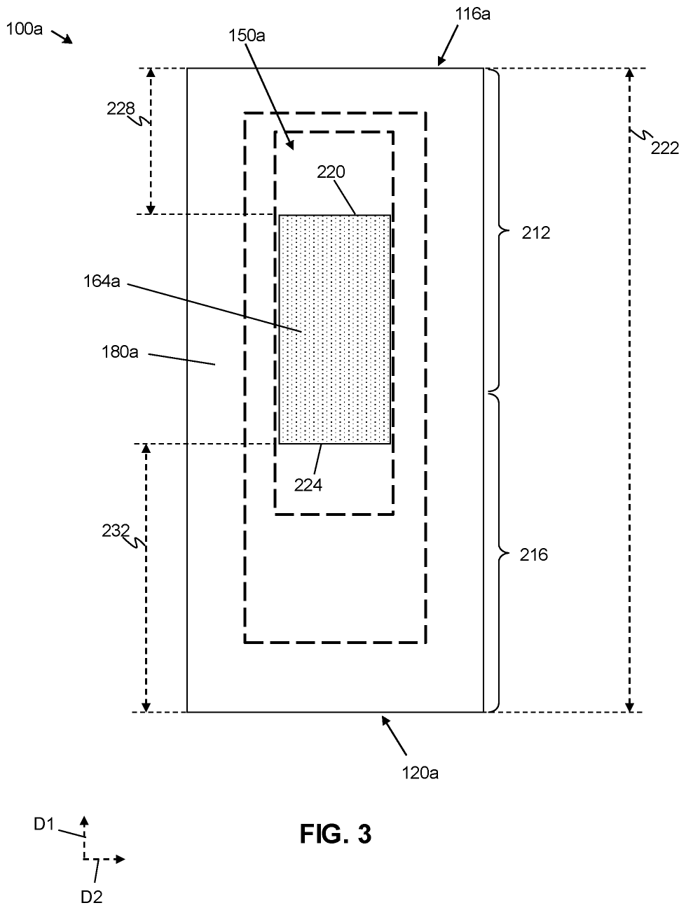

Referring now to FIG. 3, apertured region 164a need not be disposed entirely within front portion 212. In some configurations, apertured region 164a is nearer first end 116a than second end 120a. To illustrate, apertured region 164a may have a front end 220 and an opposing rear end 224, the front end being nearer first end 116a and the rear end being nearer second end 120a. A first distance 228 between front end 220 of apertured region 164a and first end 116a of chassis 104a may be less than or equal to a second distance 232 between rear end 224 of the apertured region and second end 120a of the chassis. In some such configurations, an area of apertured portion 164a disposed within front portion 212 is greater than an area of the apertured portion that is disposed within rear portion 216. Such positioning of apertured portion 164a may enable sufficient BM containment without additional modifications to prevent core leakage. Further, apertured region 164a can enable enhanced absorption of liquid insults (e.g., urine) that occur in front portion 212. In this way and others, apertured region 164a offer enhanced absorption and distribution of liquid insults at both the front and rear portions (212, 216) of chassis 104a. In some configurations, article 100a may include one or more additional features (e.g., three-dimensional voids, multi-tiered openings, or the like) positioned in rear portion 216 to increase containment of feces in the article. Such BM containment structures may be better configured to contain softened stool and lack adequate containment associated with runny BMs. Accordingly, apertured region 164a can provide increased absorption for urine as well as additional protection against runny BMs that are not adequately contained using tradition fecal containment methods.

Referring now to FIG. 3, apertured region 164a overlies an entirety of ADL 150a (e.g., the apertured region is disposed vertically above the ADL has an area that is between 90% and 110% of the area of ADL). In such configurations, a width of apertured region 164a is substantially equal to a width of ADL 150a and a length of the apertured region is substantially equal to a length of the ADL. In some such configurations, apertured region 164a and ADL 150a may be disposed entirely within front portion 212 of chassis 104a, while in other configurations, a portion of the apertured region and the ADL may be positioned within rear portion 216 (e.g., within crotch portion 132). As shown in FIG. 3, both apertured region 164a and ADL 150a have an area that is smaller than core 140a and topsheet 108a. In this way and others, material within core 140a may be contained in the core and prevented from leaking up through apertured region 164a or ADL 150a.

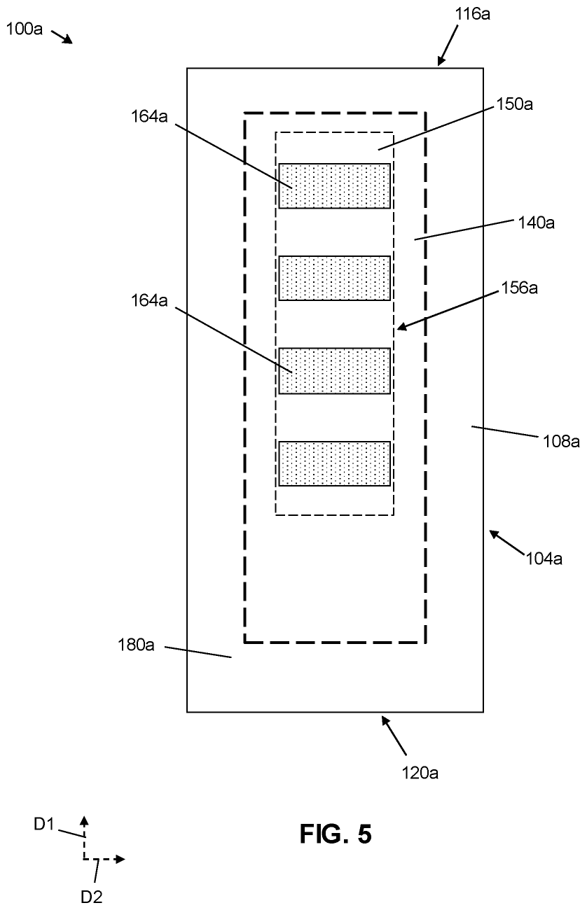

In yet other configurations, as illustrated in FIG. 5, topsheet 108a may define a plurality of apertured regions 164a. In some such configurations, each apertured region 164a overlies ADL 150a and is contained within an ADL boundary 156a. In a non-limiting illustrative example, each apertured regions 164a may have a length or width that is substantially equal to a length or width, respectively, of ADL 150a. In configurations with multiple apertured regions 164a, the apertures (e.g., 168) of topsheet 108a may be selectively positioned over portions of ADL 150a where absorption is most critical. To illustrate, one apertured region 164a may be positioned nearer first end 116a to absorb urine while another apertured region (e.g., 164a) may be positioned nearer rear portion 216 to absorb solid-fluid mixture associated with runny BM. In this way and others, apertured regions 164a can provide protection against blowouts and urine absorption while limiting the risk of SAP leakage from core 140. While depicted as being the same size, in some configurations, one apertured region (e.g., 164a) may be smaller or larger than one other apertured region. Additionally, or alternatively, apertured regions 164a may, but need not, be equally spaced along a length (e.g., 158) of ADL 150a.

The above specification and examples provide a complete description of the structure and use of illustrative embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the methods and systems are not intended to be limited to the particular forms disclosed. Rather, they include all modifications and alternatives falling within the scope of the claims, and embodiments other than the one shown may include some or all of the features of the depicted embodiment. For example, elements may be omitted or combined as a unitary structure, and/or connections may be substituted. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. An absorbent garment comprising:
a chassis including opposing front and rear waist portions, a crotch portion extending longitudinally between the front and rear waist portions, a topsheet defining a plurality of apertures, and a backsheet;
an absorbent core disposed between the topsheet and the backsheet, at least a portion of the absorbent core positioned in the crotch portion; and
an acquisition-distribution layer (ADL) disposed between the topsheet and the absorbent core, the ADL including a plurality of external edges that cooperate to define an ADL boundary;
wherein at least 75% of the plurality of apertures are defined within an apertured region that is disposed vertically above the ADL and is enclosed within the ADL boundary when the chassis is in a flat configuration.

2. The absorbent garment of claim 1, wherein the apertured region overlies at least a majority of the ADL.

3. The absorbent garment of claim 1, wherein the apertured region overlies an entirety of the ADL.

4. The absorbent garment of claim 1, wherein 95% of the plurality of apertures are defined within an apertured region

5. The absorbent garment of claim 1, wherein a width and a length of the apertured region are substantially equal to a width and a length, respectively, of the ADL.

6. The absorbent garment of claim 1, wherein:
the chassis includes:
a first end, the front waist portion extending from the first end; and
a second end opposite the first end, the rear waist portion extending from the second end; and
the apertured region is positioned closer to the first end than the second end.

7. The absorbent garment of claim 1, wherein at least a majority of the apertured region is disposed in a front half of the chassis.

8. The absorbent garment of claim 1, wherein the topsheet includes only a single apertured region.

9. The absorbent garment of claim 1, wherein the topsheet includes:
the apertured region; and
a non-apertured region surrounding the apertured region, the non-apertured region includes a percentage of open area less than two percent

10. The absorbent garment of claim 9, wherein the non-apertured region spans at least a majority of the topsheet.

11. The absorbent garment of claim 1, wherein the external edges includes: two widthwise edges extending in a first direction; and two lengthwise edges extending in a second direction perpendicular to the first direction.

12. The absorbent garment of claim 1, wherein
the topsheet includes a liquid-permeable nonwoven; and
the backsheet includes a liquid impermeable material.

13. The absorbent garment of claim 1, wherein each aperture includes a maximum transverse dimension that is between 0.25 and 5.0 mm.

14. An absorbent garment comprising:
a chassis including:
a topsheet defining a plurality of apertures and having:
an apertured region in which the topsheet includes a percentage of open area greater than or equal to 5%; and
an non-apertured region in which the topsheet includes a percentage of open area less than one percent; and
a backsheet;
an absorbent core disposed between the topsheet and the backsheet; and
an acquisition-distribution layer (ADL) disposed between the topsheet and the absorbent core, the ADL including a plurality of external edges that cooperate to define an ADL boundary;
wherein the apertured region overlies the ADL and is enclosed within the ADL boundary.

15. The absorbent garment of claim 14, wherein an area of the ADL is less than or equal to an area of the absorbent core.

16. The absorbent garment of claim 15, wherein an area of the apertured region is between 25% and 95% of the area of the ADL.

17. The absorbent garment of claim 15, wherein an area of the non-apertured region is greater than the area of the ADL.

18. The absorbent garment of claim 14, wherein:
the chassis includes:
a first end and an opposing second end;
a front portion extending from the first end; and
a rear portion extending from the second end; and
at least a majority of the apertured region is disposed within the front portion of the chassis.

19. The absorbent garment of claim 18, wherein an entirety of the apertured region is disposed within the front portion of the chassis.

20. An absorbent garment comprising:
a chassis including opposing front and rear waist portions, a crotch portion extending longitudinally between the front and rear waist portions, a topsheet, and a backsheet;
an absorbent core disposed between the topsheet and the backsheet, at least a portion of the absorbent core positioned in the crotch portion; and
an acquisition-distribution layer (ADL) disposed between the topsheet and the absorbent core, the ADL including a plurality of external edges that cooperate to define an ADL boundary;
wherein, when the chassis is in a flat configuration, the topsheet includes:
an apertured region overlying the ADL and enclosed within the ADL boundary; and
a non-apertured region; and
where an area of the apertured region is at least 90% of an area of the ADL.
